(19) United States
(12) Patent Application Publication
(10) Pub. No.: US 2017/0307365 A1
(43) Pub. Date: Oct. 26, 2017

(54) OBJECT AUTHENTICATION DEVICE AND METHOD

(71) Applicant: Paper DNA AG, Zug (CH)

(72) Inventors: Johan Bergqvist, Zug (CH); Erik Bohlin, Bromma (SE); Frank Nikolaus, Enskede (SE)

(73) Assignee: Paper DNA AG, Zug (CH)

(21) Appl. No.: 15/527,768

(22) PCT Filed: Nov. 16, 2015
(86) PCT No.: PCT/EP2015/076674

§ 371 Date: May 18, 2017

(30) Foreign Application Priority Data
Nov. 24, 2014 (CH) 01805/14

(51) Int. Cl. G01B 11/25; G06K 9/00; A61B 5/1172; G07D 7/121

(52) U.S. Cl. CPC G01B 11/25; G07D 7/121; G06K 9/00087; A61B 5/1172; G06K 9/00013; G06K 9/00046

(57) ABSTRACT

A device for authentication, which is capable of producing a topographic pattern image based on a microstructure of a paper-based artifact. The topographic pattern image of the microstructure of the paper-based artifacts is used for authentication of the paper-based artifacts. The device includes an illumination channel comprising a light source and a light director configured to bend the light beam produced by the light source. The device also includes an imaging channel comprising at least one lens configured to magnify the topographic pattern created by the light beam and an image capturing mechanism to capture the topographic pattern.

### TECHNICAL FIELD OF THE INVENTION

The present invention relates to the field of object authentication and, more specifically, relates to a device for authentication, which is capable of producing a topographic pattern image, based on the microstructure of a paper-based artifact.

### BACKGROUND OF THE INVENTION

Authentication methodologies have been used and applied mostly within the field of biometric authentication. The need to authenticate humans has been around for a long time. The events that trigger such need vary from financial transactions to entering into a foreign country, voting, giving an exam, starting up a company, etc. Biometric authentication methods use the inherent and unique characteristics of individuals. Fortunately, human beings consist of complex organic systems that have high degrees of uniqueness. The most common organic feature utilized for authentication purposes has been fingerprints. More advanced methods can use the iris of the human eyes, blood vessel patterns hidden under the skin of the hand and the face. Other methods involve the use of voice. Most of these methods are based on image processing and recognition. These are possible because of the great diversity of features in the human body.

However, in a plain document or a common package, such great diversity does not exist. As a matter of fact, two documents or two packages of the same size look completely identical to one another at the first sight. With naked eye, there is no obvious alternative to differentiate two identical documents in the absence of any specific marks printed or written on them.

However, there is a less obvious although extremely robust alternative to differentiate, not only apparently identical sheets of paper or packages but also small specific areas within the same paper sheet or the same package.

It is known through various research studies and through existing inventions in the same field that the inherent microstructure of paper-pulp based product is so unique that a couple of square centimeters have enough details to be able to create a very robust authentication methodology.

The U.S. Pat. No. 7,853,792 discloses a method and apparatus for determining a digital signature from an article made of paper, cardboard, plastic or many other material types. A coherent light source directs a beam to illuminate the article and a detector arrangement collects data points from light scattered from many different parts of the article to collect a large number of independent data points, typically 500 or more. By collecting a large number of independent signal contributions specific to many different parts of the article, a digital signature can be computed that is unique to the area of the article that has been scanned. The principal optical components are a laser source for generating a coherent laser beam.

The United States Patent Application No: 20100007930 discloses a system for obtaining a signature from a scan area on the surface of an article comprising a signature generator that generates the signature from scattered coherent radiation detected from a plurality of points on the surface and includes a scan head comprising a coherent radiation source and photo detectors, a camera for capturing an image of the surface, a comparator that compares the captured image with a reference image to determine the location and orientation of the scan area, and a drive assembly that positions the scan head appropriately for generating a signature from the scan area in response to the determination of the location and orientation of the scan area.

Most of the existing prior arts are based on the use of laser equipment as the source for illumination. These inventions utilize the scattering effect of the laser light. A drawback of such method is that such encoding and decoding devices become rather bulky and expensive. The success of an authentication procedure is that a decoding device can be made portable and inexpensive so that broad adoption can be achieved.

Hence, there is a need for a portable device, which can be used for authentication of paper-based artifacts, wherein the cost of manufacture of such a device is minimal.

### SUMMARY OF THE INVENTION

The invention provides a device for authentication, which is capable of producing a topographic pattern image based on a microstructure of a paper-based artifact, the topographic pattern is created by illuminating the paper-based artifact from an angle. The topographic pattern image of the microstructure of the paper-based artifacts is used for authentication of the paper-based artifacts. The device is characterized by an illumination channel comprising a light source and a light director configured to bend the light beam produced by the light source. The device also includes an imaging channel comprising at least one lens configured to magnify the topographic pattern image created by the light beam and an image capturing mechanism to capture the magnified topographic pattern image.

The device is further characterized by a mechanical aperture below the imaging channel. The device further has a protective cap, wherein the protective cap is removeably attached to a lower part of the device, wherein the protective cap is configured to protect the light director and lens from stains, dust and other contaminants by closing the mechanical aperture.

According to another embodiment, the device is further characterized by a protective gliding cap, wherein the protective gliding cap is configured to be pulled back to allow the optical parts to become functional over the working surface.

All elements of the present invention are widely used components and therefore quite affordable. Thus, the aforesaid device may be manufactured cost effectively. It is envisaged that the device described in the present invention may be used either as an encoding module in which, the image captured by the module would be used to characterize and codify the pattern created by the microstructure in that particular area, or as a decoding module in which the image captured by the device would be to check that the observed pattern belongs to the microstructure that has been previously codified.

### BRIEF DESCRIPTION OF THE DRAWINGS

The foregoing and other features of embodiments will become more apparent from the following detailed description of embodiments when read in conjunction with the accompanying drawings. Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention. Thus, in the interest of clarity and conciseness, the drawings are generalized in form, wherein:

FIG. 1 illustrates a cross sectional view of a device, in accordance with an embodiment of the invention.

FIG. 2 illustrates the bottom view of the device, in accordance with an embodiment of the invention.

FIG. 3 illustrates the protective cap of the device, in accordance with an embodiment of the invention.

FIG. 4 illustrates an isometric view of an authentication device in accordance with an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable a person skilled in the art to practice the invention, and it is to be understood that other embodiments may be utilized and that logical, mechanical, and other changes may be made within the scope of the embodiments. The following detailed description is, therefore, not to be taken as limiting the scope of the invention, but instead the invention is to be defined by the appended claims.

The present invention discloses a device for authentication, which is capable of producing a topographic pattern image based on a microstructure of a paper-based artifact. The topographic pattern image of the microstructure of the paper-based artifacts is used for authentication of the paper-based artifacts. The device is characterized by an illumination channel comprising a light source and a light director configured to bend the light beam produced by said light source. The device also includes an imaging channel comprising at least one lens configured to magnify the topographic pattern created by the light beam and an image capturing mechanism to capture said topographic pattern.

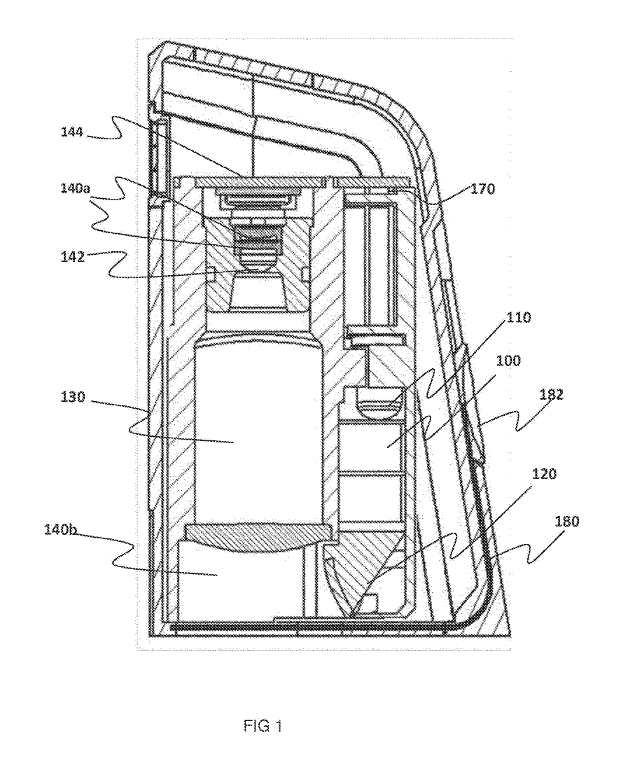

According to an embodiment of the invention, FIG. 1 illustrates the cross sectional view of the device for authentication. The device for authentication is capable to produce a topographic pattern image based on a microstructure of a paper-based artifact. The topographic pattern image of the microstructure of said paper-based artifacts is used for authentication of the paper-based artifacts. The device is characterized by an illumination channel (100) comprising a light source (110). The light source (110) may be a light emitting diode. The illumination channel (100) further includes light director (120) configured to bend the light beam produced by the light source (110). The light beam from the illuminating channel (100) is bent so that the light beam may fall on the paper-based artifact at certain angle. The light director (120) may be a prism or a mirror which bends the light falling on the surface so that the light is directed to fall on the paper-based artifact at certain angle. The prisms have the function to bend the light beam from the light source (110) in a certain angle necessary to create a visible pattern out of the inherent irregularities of the paper microstructure. Such pattern would be a combination of bright tops and shadows produced by the paper-pulp fibers which originally have irregular cylindrical long shape.

The device may also include an imaging channel (130) comprising one or more lens (140). The lens (140) may be one of more field lenses, one or more objective lenses configured to magnify the topographic pattern created by the light beam, as a result the paper-based artifact's microstructure is clearly visible. The imaging channel (130) has two objective lens (140a) and a tele centric lens (140b). The imaging channel (130) further includes an image capturing mechanism (144) to capture the topographic pattern image created. The image capturing mechanism (144) may be a complementary metal-oxide-semiconductor (CMOS) imaging integrated circuit (IC) and a microcontroller unit. The purpose of the imaging channel (130) is to optically enlarge certain portion of the paper-based artifact to be authenticated which is illuminated as described above. The topographic pattern image of such enlarged portion is then captured by the CMOS imaging integrated circuit through the optical aperture (142). Through various imaging processing methods, the topographic pattern image may be used to codify the particular and unique pattern in the microcontroller unit for future recognition and matching. The various lenses present in the imaging channel (130) along with the light director (120) are the various optical parts present in the device. The light flash required for the CMOS camera to acquire useful images is perfectly triggered to the capture event and exposure time is programmed so that no light and energy is wasted. Light should be switched off during image processing time. The device further has a PCB (printed circuit board) (170) and a power source for the functioning of the device. The device further has a protective gliding cap (180). The protective gliding cap (180) is configured to be pulled back to allow the optical parts to become functional over the working surface. The protective gliding cap (180) is configured to be pulled back to allow the optical parts to become functional over the working surface. The protective gliding cap (180) may be pulled back and pushed forth by means of a switch (182). This allows the protection of the imaging channel (130) from dust and other dirt when the device is not in use.

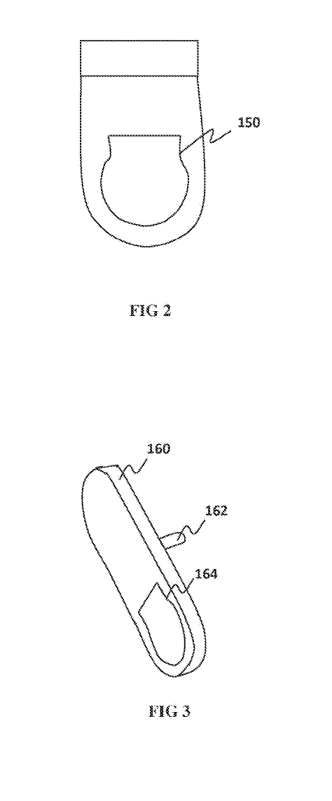

FIG. 2 illustrates the bottom view of the device, in accordance with an embodiment of the invention. The mechanical aperture (150) is used for exposing the paper-based artifact to the device for capturing the topographic pattern image. The mechanical aperture (150) is kept closed when the device is not in use so as to protect the optical parts of the device from dust and other stains or contaminants. The device is placed over the paper-based artifact so that the imaging channel (130, FIG. 1) is positioned above the paper based artifact through the mechanical aperture (150).

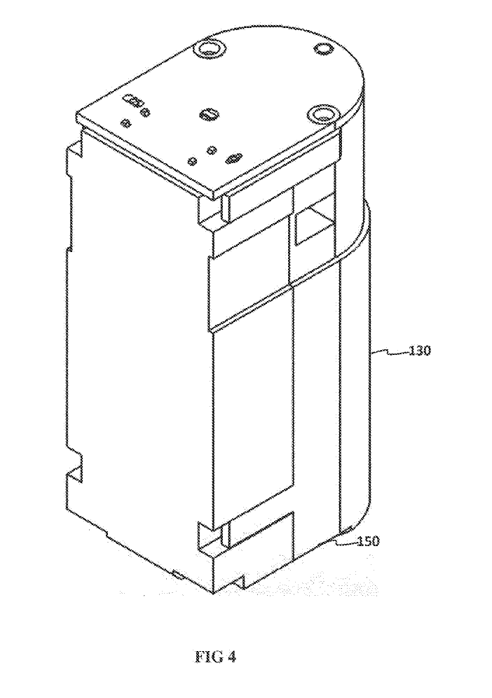

In a preferred embodiment, the device is further characterized by a protective cap (160, FIG. 3), wherein the protective cap is removeably attached to a lower part of the device. The protective cap is configured to protect the optical parts from stains, dust and other contaminants.

FIG. 3 illustrates a protective cap, in accordance with an embodiment of the invention. The protective cap (160) is removeably attached to the bottom of the device. The protective cap (160) rotates on the rotation axis (162) between two positions. In one position, the protective cap closes the mechanical aperture (150, FIG. 2) and in another position the protective cap (160) opens the mechanical aperture (150, FIG. 2) of the device by aligning an aperture (164) of the protective cap (160) along with the mechanical aperture (150, FIG. 2)

FIG. 4 illustrates the isometric view of a device, in accordance with an embodiment of the invention. This figure illustrates the device assembled with the illuminating channel and imaging channel (130) with outer covering. The mechanical aperture (150) is in the lower bottom portion of the device.

According to an embodiment of the invention, the device may have a power source such as rechargeable battery to function. The rechargeable battery may be charged using a docking station.

It is envisaged that the device, described in the present invention may be used either as an encoding module in which, the image captured by the module would be used to characterize and codify the pattern created by the microstructure or as a decoding module in which the image captured by the device would be to check that the observed pattern belongs to the microstructure that has been previously codified. Various techniques of encoding and decoding may be utilized along with the device to perform authentication of the object.

According to an embodiment of the invention, the topographic image captured by the device is codified to generate an authentication mark by an authentication encoder module. The authentication encoder module may work along with a printing system by directly printing the authentication mark into packages and documents or using a label.

The authentication encoder module has a very compact size and may be implemented in any type of production- or assembly line. It may also be built into existing equipment including scanners, printers, multi functional printer, and digital presses. Every authentication encoder contains a 128-bits cryptographic key that is transferred to the authentication marks for handshaking with the decoder. This makes production of fraudulent equipment very difficult.

All elements of the present invention are widely used components and therefore very affordable. Thus, the aforesaid device may be manufactured cost effectively. In such cases the optical lenses may be made in cost-effective thermo polymer material. The CMOS imaging IC are very similar or identical to those widely used in smartphones, tablets and digital cameras. Due to high volumes, they have become a commodity. Light emitting diodes are also a commodity as they are found everywhere: Household and office lightning, street lightning, in vehicles of all kinds, decorative lightning, back displays and much more

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description.

1. A device for authentication, capable of producing a topographic pattern image based on a microstructure of a paper-based artifacts, wherein the topographic pattern image of the microstructure of said paper-based artifacts is used for authentication of the paper-based artifacts, the device comprising:
a. an illumination channel comprising a light source of at least one light emitting diode, and a light director configured to bend the light beam produced by said light source, whereby illuminating the paper-based artifacts at certain angle; and
b. an imaging channel comprising of at least one lens configured to magnify the said microstructure created by the light beam, whereby the paper-based artifact's microstructure is clearly visible and an image capturing mechanism to capture said topographic pattern image.

2. The device as claimed in claim 1, the device further comprising a mechanical aperture below the imaging channel on the lower part of the device.

3. The device as claimed in claim 1, the device further comprising a protective cap, wherein the protective cap is removeably attached to a lower part of the device, wherein the protective cap is configured to protect the light director and lens from stains, dust and other contaminants by closing the mechanical aperture.

4. The device as claimed in claim 1, the device further comprising a protective gliding cap, wherein the protective gliding cap is configured to be pulled back to allow the optical parts to become functional over the working surface.
